(19) United States
(12) Patent Application Publication
(10) Pub. No.: US 2019/0164631 A1
(43) Pub. Date: May 30, 2019

(54) BIOMARKERS SIGNATURE DISCOVERY AND SELECTION

(71) Applicant: SimplicityBio SA, Monthey (CH)

(72) Inventors: Miguel BARRETO-SANZ, Lausanne (CH); Carlos Andrés PEÑA REYES, Vicques (CH)

(73) Assignee: SimplicityBio SA, Monthey (CH)

(21) Appl. No.: 16/098,817

(22) PCT Filed: May 19, 2016
(86) PCT No.: PCT/IB2016/052936

§ 371 Date: Nov. 2, 2018

(51) Int. Cl. G16B 40/00; G16B 20/00; G06N 7/02

(52) U.S. Cl. CPC G16B 40/00; G06N 7/02; G16B 20/00

(57) ABSTRACT

The present invention concerns a method for discovering at least a biomarkers signature from biomarker data pools, the method comprising the steps of: i) Generating a set of signatures (1, 2) with fuzzy logic and evolutionary algorithms, each signature reciting a determined number of biomarkers; ii) Selecting at least a target signature from said set of signatures by applying at least the following filters on said set of signatures (1, 2): a) a performance filter; b) a frequency filter; and c) a dominance filter. The present invention further relates to device for discovering at least a biomarkers signature from biomarker data pools and to a use of a biomarker signature discovered by a method according to the present invention for diagnosing, predicting or monitoring a disease.

### FIELD OF THE INVENTION

The present invention concerns a method for discovering biomarkers signature, a device, a use and a computer program product related thereof.

### DESCRIPTION OF RELATED ART

In the biomedical field, there is a constant need to identify biomolecules (proteins, nucleic acids for instance) or physiological parameters called biomarkers, that are indicative of a specific biological status. Biomarkers are not only useful for diagnosis and prognosis of many diseases, but also for understanding the basis for development of therapeutics. Successful and effective identification of biomarkers can accelerate new drug development process.

Recent technologies of genomics and proteomics emergences, including high throughput screening, supplies a wealth of information regarding biomarkers, such as numbers and forms of proteins expressed in a cell. It is possible to identify for each cell, a profile of expressed proteins characteristic of a particular patient status, either sick or healthy status. Additional information are provided by experimental measurements of physiological parameters of the patient for instance blood pressure, weight or cardio/renal related data.

Consequently, comparing biomarkers input from a patient with a disease to that of a healthy patient can provide opportunities to identify a set of biomarkers, called a signature, that are relevant for diagnosing, monitoring, prognosis or predicting a disease. In this respect, several computer-based methods have been developed to identify signatures that best discriminate a sample from a sick patient from the one of a healthy patient.

For instance, the document WO2013190086 teaches a method combining Significance Analysis of Microarrays (SAM) analysis and Limma analysis or Matthew correlation to generate a signature.

Alternatively, the document EP0827611 discloses a method to generate signatures based on fuzzy logic to identify biomarkers chosen amongst cells pools, regulators, chemical production, human or anatomical response and manifestation of the disease at different level or hierarchy.

The document PENA-REYES, Carlos Andres, “Coevolutionary fuzzy modelling” (2002), teaches a method to provide a biomarkers signature with fuzzy logic and genetic algorithm from biomedical data. Fuzzy logic is based on the assumption that a statement may be partially right (or false) in contrast with a Boolean system. Fuzzy logic is particularly suitable for processing biomedical data by allowing a more accurate description of the evolution of a medical status, for instance from a healthy to a sick status. Fuzzy logic permits to take into account the variations and the intermediate levels of a status whereas Boolean system would focus on arbitrary status, either sick or healthy for instance. In the document PENA-REYES, fuzzy logic is combined with genetic algorithm. Genetic algorithm is a well-recognized technique that takes into account the natural evolution of genetic information, in particular the Darwinian principle of survival of the fittest.

Classically, the first step to identify an accurate signature concerns the generation of a set of signatures comprising up to several thousands of signatures, each signature generally reciting several dozen or hundreds of biomarkers. It is necessary to generate several signatures to optimize the chance of identifying at least one accurate signature. Later on, a user analyses and compares the generated signatures among the set to select one or more of them. It is possible to provide satisfying results with a manual method if the set of signatures to be sorted out is limited, typically below a dozen signatures.

However, when it comes to sorting out the most accurate signature among a set of hundreds or thousands, the manual selection process is not adapted any more, mainly because this process is very time consuming. Moreover, the manual selection is error prone when the number of signature to be sorted is important.

Therefore, there is a need for a method to sort out at least a target signature among a set of signature in an efficient and accurate manner.

### BRIEF SUMMARY OF THE INVENTION

One of the aim of the invention is to provide a method for discovering biomarker signature free from, or at least minimizing, the limitations of the known methods.

Another aim of the invention is to provide a method for discovering at least one signature in an accurate and efficient manner in particular when the set of signatures comprises a great number of signatures, for instance above twenty to fifty signatures.

According to the invention, at least a part of these aims are achieved by means of a method for discovering at least a biomarker signature from biomarker data pools, the method comprising the steps of:
i) Generating a set of signatures with fuzzy logic and evolutionary algorithms, each signature reciting a determined number of biomarkers;
ii) Selecting at least a target signature from said set of signatures by applying at least the following filters on said set of signatures:
   a) a performance filter comprising:
      setting a performance threshold for at least a performance criterion;
      removing signature having a value below said performance threshold for said performance criteria;
   b) a frequency filter for sorting said set of signatures depending on the frequency of one biomarker within said set of signatures or the co-frequency of several biomarkers within the same signature among said set of signatures; and
   c) a dominance filter comprising:
      ranking the set of signatures depending on at least one performance criteria;
      computing a dominance value for each signature, the dominance value being the number of signatures with a superior ranking for said performance criteria;
      setting a dominance threshold and removing the signatures having a dominance value higher than said dominator threshold.

The method according to the present invention allows sorting out at least a target signature among a set of signatures. The inventors discovered that by applying at least one performance filter, at least one frequency filter and at least one dominance filter to a set of signatures generated by fuzzy logic and genetic algorithm, it is possible to select at least one target signature in a more efficient and accurate manner than with the existing methods.

The present invention allows providing robust target signatures because the target signatures are selected in a soft, fine-tuned and stepwise approach.

In the present invention, the use of at least a performance filter, a frequency filter and a dominance filter have a synergic effect, meaning that the selection of target signatures is more efficient by using at least a performance filter, a frequency filter and a dominance filter than when one of said filters is applied individually on the set of signatures.

Advantageously, the present invention allows minimizing the number of biomarker in the target signature while maintaining exceptional results, for instance in terms of accuracy, sensitivity and specificity. Similarly, the method according to the present invention also permits to minimize the number of rules in the target signature.

By constraining the number of rules and biomarkers in each of the signature, testing costs based on the target signature will be reduced, both on the development end and consumer end. A cleaner, more concise target signature can also aid developers in navigating the regulatory approval process likely to follow the discovery of a target signature.

Performance filter is a filter that sorts out the signatures based on a threshold value of a performance criteria in learning. For instance, the performance criteria is chosen among specificity, sensitivity, accuracy, positive and negative predictive values (PPV and NPV respectively), number of biomarkers or rules per signature, area under the ROC curve (AUC), and average distance measurement (ADM).

Sensitivity is a parameter focusing on sick people by describing the proportion of true positives, i.e. sick people, that are correctly identified as such among those who have the disease. Sensitivity is defined as:

Sensitivity: TruePos/(TruePos+FalseNeg)

Specificity is a parameter concerning healthy people by describing the proportion of true negatives, i.e. healthy people, that are correctly identified as such among those whose are healthy. Specificity is defined as:

Specificity: TrueNeg/(TrueNeg+FalsePos)

In the present invention, the accuracy is a parameter that takes into account the specificity and the sensitivity, said accuracy being defined as:

Accuracy: (TruePos+TrueNeg)/(TruePos+TrueNeg+FalsePos+FalseNeg)

Positive and negative predictive values (PPV and NPV respectively) concerns the proportions of positive and negative results that are true positive and true negative results. PPV is defined as:

PPV: TruePos/(TruePos+FalsePos)

NPV is defined as:

NPV: TrueNeg/(TrueNeg+FalseNeg)

In one embodiment, the application of one frequency filter comprises:
selecting at least one biomarker listed in the set of signatures;
removing the signature(s) free from said selected biomarker.

For instance, if the frequency filter is set on a biomarker A, then, all the signature comprising the biomarker A will be selected. Similarly, the frequency filter can also be used to select the co-frequency of two or more biomarkers within one signature.

According to an embodiment, the application of one frequency filter comprises:
computing a frequency of each biomarker in the set of signatures;
defining a frequency threshold for at least one biomarker;
removing the signature(s) comprising biomarker(s) with a frequency below said frequency threshold.

For instance, in this embodiment the frequency filter allows removing the signatures comprising biomarkers little used in the set of signatures.

The inventors found out that the dominance filter is an efficient filter to provide accurate target signatures. The dominance filter is used to compare the signatures depending on at least one performance criteria. First, the signatures are ranked depending on at least a performance criteria, said performance criteria can be the same than the performance criteria used in the performance filter or a different one. Then, a dominance threshold is set and the signature are sorted out by comparing their respective dominance value with the dominance threshold: if a signature has a dominance value above the dominance threshold, said signature is removed.

For instance, a dominance filter uses the sensitivity and the specificity as performance criteria. The set of signatures to be sorted out is the following:
Signature A: specificity 80%, sensitivity 90%
Signature B: specificity 90%, sensitivity 80%
Signature C: specificity 60%, sensitivity 60%
   In the present example, the dominance values are the following:
Signature A: 1 (dominated by B in specificity)
Signature B: 1 (dominated by A in sensitivity)
Signature C: 2 (dominated by A and B in specificity; dominated by A and B in sensitivity)

In the present case, the signature C is always dominated by two signatures, either in sensitivity or in specificity. A dominance threshold is set to two, meaning the all the signatures being dominated by two or more signatures, called dominators, is removed. Thus, signature C is removed from the set of signatures.

The dominance filter allows sorting out the set of signatures by comparing signatures to each other's: a signature is selected if said signature dominates “X” other signatures (“X” being the dominance threshold). On the contrary, with the performance filter for instance, a signature is selected if said signature has a performance value above a threshold. The advantage of the dominance compared with other filters is that it allows to select several good alternatives. Each option is first assessed under multiple criteria and then a subset of options is identified with the property that no other option can categorically outperform any of its members. By yielding all of the potentially optimal solutions, the selection can make focused trade-offs within this constrained set of parameters, rather than needing to consider the full ranges of parameters.

In one embodiment, the performance criteria of the dominance filter are the specificity and the sensitivity.

In one embodiment, the performance criteria of the dominance filter is the specificity.

In one embodiment, the performance criteria of the dominance filter is the sensitivity.

In one embodiment, the performance criteria of the dominance filter is the accuracy.

In another embodiment, the performance criteria used in the performance filter are the sensitivity and the specificity.

In one embodiment, the target signature is selected by using successively at least one performance filter, at least one frequency filter and at least one dominance filter.

According to an embodiment, step ii) comprises:
applying several sequence of filters on the set of signatures, each filter sequence comprising at least one filter, each filter sequence being applied separately on the set of signatures so that each filter sequence provides at least one preselection of signature;
identifying at least one common signature, said common signature being at least one signature present in all the preselection;
combining the common signature(s);
applying at least one filter on the common signature(s).

A sequence of filters comprises at least one filter. In this embodiment, several sequences of filters are applied separately, i.e. in parallel, meaning that each sequence of filter is applied on the set of signature to provide one preselection of signature. Each preselection comprises a determined number of signatures. When one specific signature is listed in several preselections, said signature is designated as a common signature. The common signatures are combined and subsequently filtered.

In one embodiment, step ii) comprises the successive steps of:
applying a first performance filter on the set of signatures;
applying at first frequency filter on the signature isolated in the previous step to provide a first preselection of signature;
applying a first dominance filter independently on the signature isolated by the first performance filter to provide a second preselection of signature;
combining the signature listed in both the first preselection and the second preselection;
applying a second dominance filter on the signature isolated in the previous step;
applying a second performance filter on the signature isolated in the previous step;
applying a third performance filter on the signature isolated in the previous step;
applying an expert filter on the signature isolated in the previous step.

In this embodiment, two sequence filters are applied on the set of signatures, a first filter sequence comprising at least a frequency filter providing a first preselection and a second filter sequence comprising at least a dominance filter providing a second preselection. Signatures selected in both the first preselection and the second preselection are combined and filtered subsequently. The inventors found out that this embodiment provides reliable target signatures, because each target signature is selected by two independent sequence filters.

In one embodiment, several iterations of the method are performed, each iterations providing a family of target signatures. A family of target signatures can be provided to meet the specific needs of a client, for instance, some families are focused on sensitivity, others have a limited number of biomarkers. A family of target signatures gathers at least two target signatures with a special feature. A family of target signature can be generated by one iteration of the method according to the invention. Several families of target signature can be generated by running several iteration of the method according to the present invention.

In one embodiment, the target signature(s) comprises at least one rule. The rule(s) permits to define the relationship between the biomarkers.

According to an embodiment, the method further comprises an expert filter applied by an expert in signature discovery to remove at least one biomarker from the target signature. For instance, the expert filter is used as a last step of the method, to fine tune the target signature. For instance, the expert filter is used to remove at least an irrelevant variable that remains after artificial evolution. The expert filter can also be used to choose in favour of a defined biomarker to meet a client request.

According to an embodiment, the data pools comprises data chosen amongst protein or nucleic acid measurements, physiological parameters such as age, weight, gender, or other clinical data. For instance, the data pools comprise plasma/blood concentrations of biomolecules, or measurements of physiological parameters of healthy and sick patients.

In one embodiment, the data pools comprise biomedical data from sick and from healthy patients. In particular the data pools comprise biomedical data from sick and from healthy human patients. The data pools can also comprise biomedical data from patients developing certain disease. The data pools can also comprise biomedical data from patients developing from patients at different disease stages. One or several pool(s) can comprise data from healthy patients and (an) other(s) pool(s) can comprise data from sick patient.

In one embodiment, the data pools comprises data from sick and from healthy plants. Thus, the method according to the present invention can be used with plants to discover biomarker signature comprising plants' biomarkers. The data pools from plants can comprise data from any healthy or sick plant, in particular crops for food production human or animal (for instance corn, soya etc), textile production (including cotton, Corchorus genus, Linum usitatissimum etc), plants used to create biofuels (including wheat, corn, sugar beets, sugar cane etc), medicinal plants (aloe vera, Wild Ginger, Belladonna etc), for production of alcoholic beverages (including vineyard, blue agave etc).

In one embodiment, the data pools comprises data from sick and from healthy animal. Thus, the method according to the present invention can be used with animals to discover biomarker signature comprising animals' biomarkers. The data pools from animals can comprise data from any healthy or sick animals, domestic animals or wild animals. In particular, animals are used for food production (chickens, fish, bees, cows), domestic animals (cats, dogs etc), animal in sport (including horses, dogs etc) and animals from pre-clinical studies.

Another aim of the invention is to provide a device for discovering at least a biomarkers signature from biomarker data pools free from the limitations of the known device.

According to the invention, this aim is achieved by means of a device for discovering at least a biomarkers signature from biomarkers data pools, the device comprising:
A system combining fuzzy logic with evolutionary algorithms for generating a set of signatures, each signature reciting a determined number of biomarker;
A filter system for selecting at least a target signature from said set of signatures, said filter system comprising at least the following filters:
   a performance filter for sorting said set of signatures depending on at least one performance criteria;
   a frequency filter for sorting said set of signatures depending on the frequency of one biomarker within said set of signatures or the co-frequency of several biomarkers within the same signature among said set of signatures;
   a dominance filter, each signature has a dominance value so that the dominance filter is capable of sorting said set of signatures depending on the dominance value.

The invention further concerns a use of a biomarker signature discovered by a method according to the present invention for diagnosing, predicting or monitoring a disease.

In one embodiment, the disease is chosen among plant disease, human disease, animal disease.

The embodiments regarding the method according to the present invention apply mutatis mutandis to the device and to the use according to the present invention, and vice versa.

A method, a device or a use according to the present invention can comprise an isolated embodiment.

A method, a device or a use according to the present invention can comprise a combination of a plurality of embodiments.

In the context of the invention, the terms “biomarker” is defined as a characteristic that is objectively measured and evaluated as an indicator of normal biologic processes, pathogenic processes, or pharmacologic responses to a therapeutic intervention, as defined by the National Health Institute (NIH, USA). A biomarker can be a biomolecule, such as protein or nucleic acids, or a physiological parameter (blood pressure for human or animal) of a human, an animal or a plant.

In the context of the invention, the terms “signature” or “biomarker signature” are interchangeable and synonym. The terms “signature” or “biomarker signature” refer to at least two biomarkers that are relevant to describe a particular status of a patient.

In the context of the invention, a target signature can comprise several rules. The term “rule” describes the variation of the biomarker of a signature. In one embodiment, the target signature(s) comprises at least one rule.

For instance, if the signature comprises three biomarkers (BM), namely BM1, BM2, BM3, rules 1 and 2 teach that:

Rule 1: if (BM1 is Low) and (BM2 is Low) and (BM3 is Low) then (the patient is sick)
Rule 2: if (BM1 is high) and (BM2 is Low) and (BM3 is high) then (the patient is sick)

The terms high and low refers for instance to the plasma or blood concentration of biomarker with respect to one or several threshold(s) when the biomarkers are a biomolecule.

On the context of the invention, the term “filter” refers to a mathematical operation allowing to remove at least one signature from the set of signatures.

### BRIEF DESCRIPTION OF THE DRAWINGS

The invention will be better understood with the aid of the description of two embodiments given by way of examples and illustrated by the figures, in which:

FIG. 1 shows the filters used in a first and second embodiment of the present invention;

FIGS. 2 and 3 illustrate the first embodiment the present invention focusing on a colon cancer study;

FIGS. 4 and 5 illustrate the second embodiment the present invention focusing on a prostate cancer study;

### DETAILED DESCRIPTION OF POSSIBLE EMBODIMENTS OF THE INVENTION

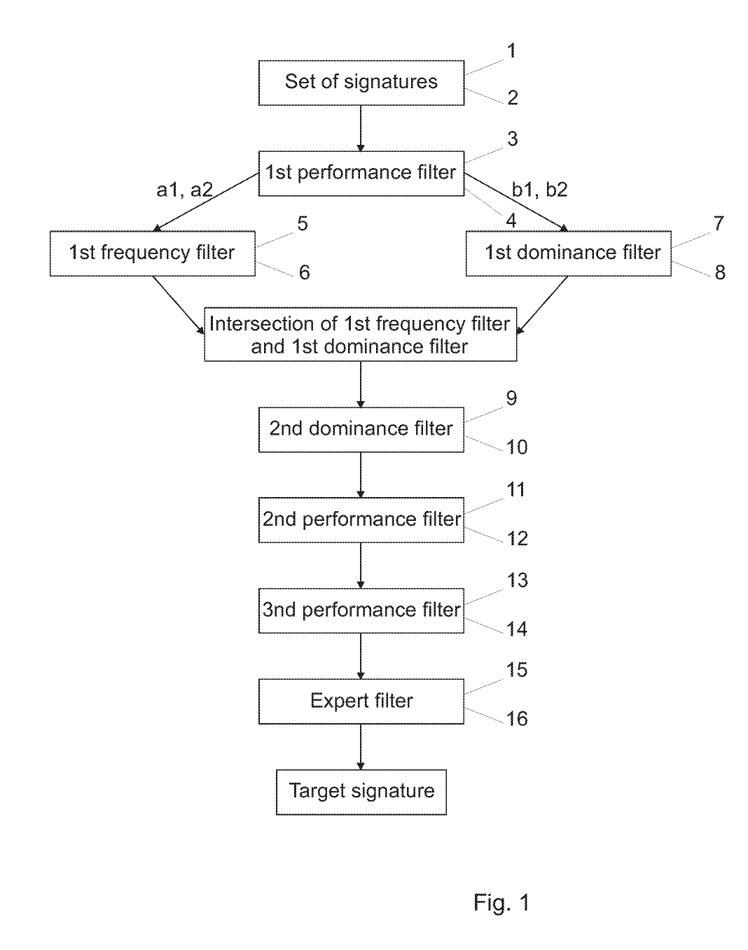

FIG. 1 illustrates the filters used in a first and second embodiment of the present invention concerning respectively signatures for human colon cancer diagnosis and human prostate cancer diagnosis but it is intended that the invention is not limited to human disease, the invention can also be applied to plant or animal disease by using respectively plant or animal biomarker database pools.

In the first and second embodiment, the method for signature discovery starts with a first performance filter 3, 4. Then, the method comprises two sequences of filter:
a first sequence filter a1, a2 comprising a first frequency filter 5, 6;
a second sequence filter b1, b2 comprising a first dominance filter 7, 8;

Then, the signatures selected both in the first sequence filter a1, a2 and in the second sequence filter b1, b2 are combined and a second dominance filter 9, 10 is applied. Subsequently, a second performance filter 11, 12 and a third performance filter 13, 14 are applied successively. Eventually, an expert filter 15, 16 allows providing the target signature.

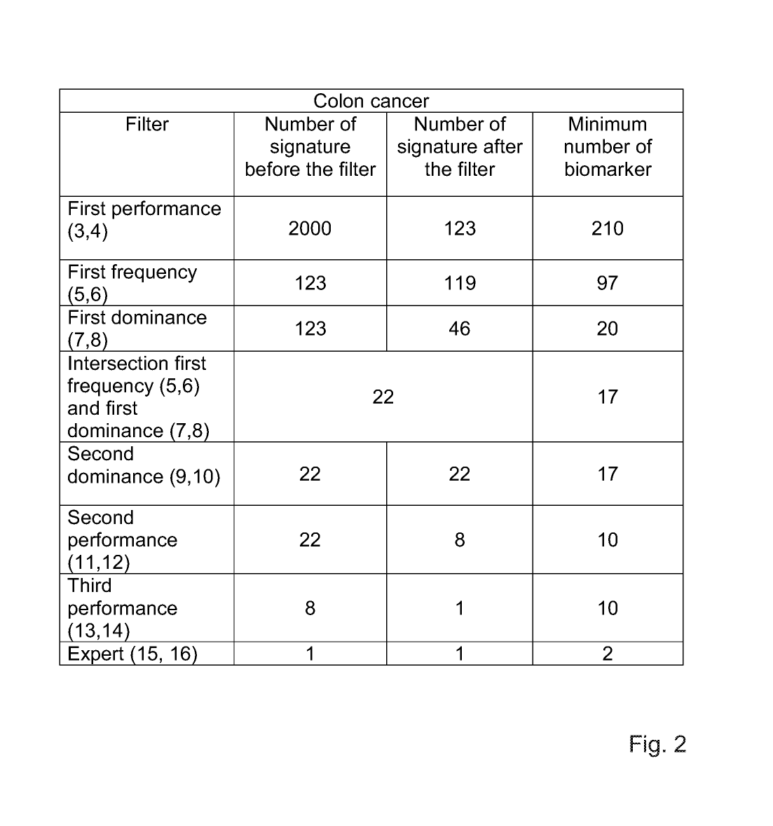

The first embodiment aims at discovery a colon cancer target signature. To that end, a data pool of 40 tumors samples and 22 normal samples is analysed, each sample comprising 6000 genes (Alon, U., Barkai, N., Notterman, D. A., Gish, K., Ybarra, S., Mack, D., & Levine, A. J. (1999). Broad patterns of gene expression revealed by clustering analysis of tumor and normal colon tissues probed by oligonucleotide arrays. Proceedings of the National Academy of Sciences, 96(12), 6745-6750.). A set of 2000 signatures is then generated by using fuzzy logic and genetic algorithm. The set of signatures is sorted out by using the filters illustrated in FIG. 1 (references 1, 3, a1, 5, b1, 7, 9, 11, 13, 15 of FIG. 1). The results obtained in this first embodiment are showed in FIG. 2. The method according to the present invention allows selecting a target signature comprising 2 biomarkers starting from a set of 2000 signatures. The method also permits to decrease drastically the number of biomarkers in the signature from 210 biomarkers to 2 biomarkers for the target signature.

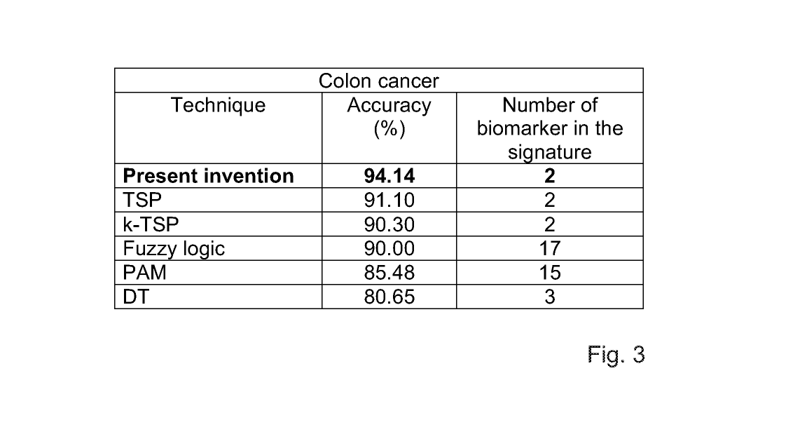

The accuracy of the target signature in the diagnosis of colon cancer was compared to the accuracy provided by other existing techniques, as shown in FIG. 3. The present invention proved to be the best method with an accuracy of 94.14% by using a target signature with only 2 biomarkers.

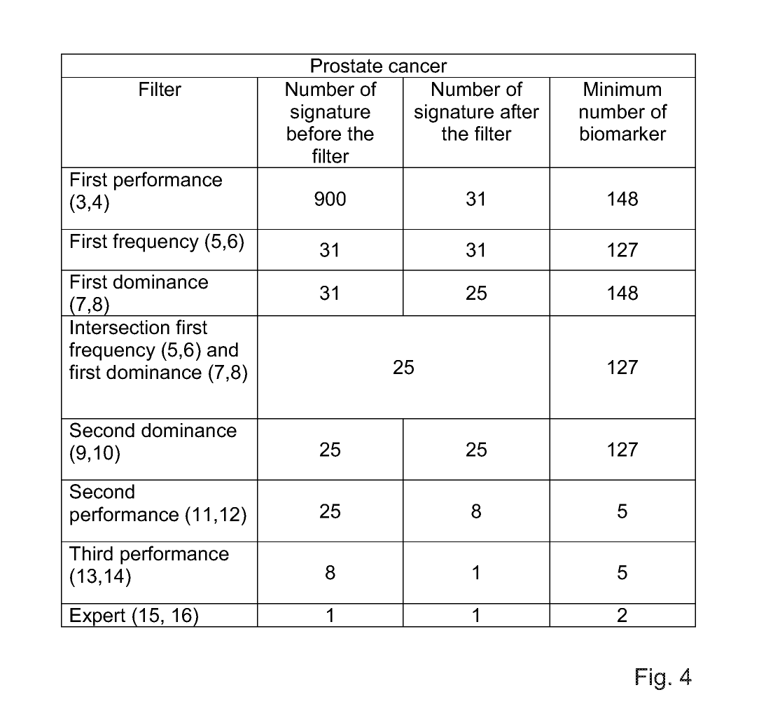

The second embodiment aims at discovering a prostate cancer target signature. To that end, a data pools of 52 tumors samples and 50 normal sample were analysed, each samples comprising 12'600 genes (Singh, D., Febbo, P. G., Ross, K., Jackson, D. G., Manola, J., Ladd, C., . . . & Sellers, W. R. (2002). Gene expression correlates of clinical prostate cancer behavior. Cancer cell, 1(2), 203-209). A set of 900 signatures is generated by using fuzzy logic and genetic algorithms. The set of signatures is sorted out by using the filters illustrated in FIG. 1 (references 2, 4, a2, b2, 6, 8, 10, 12, 14, 16 of FIG. 1). The results obtained in this first embodiment are showed in FIG. 4. The method according to the present invention allows selecting a target signature comprising 2 biomarkers starting from a set of 900 signatures. The method also permits to decrease drastically the number of biomarkers in the signature from 148 biomarkers to 2 biomarkers for the target signature.

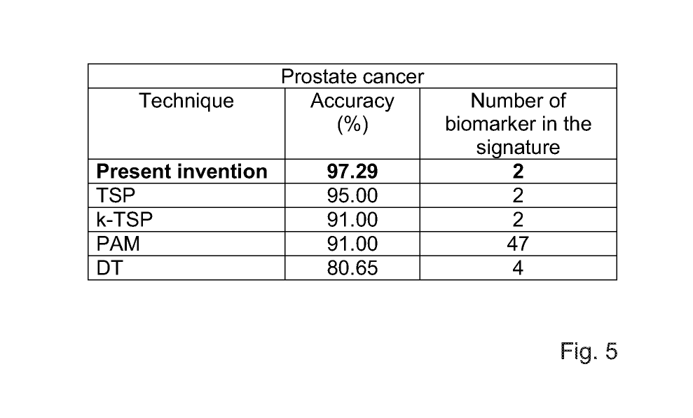

Similarly to the first embodiment, the accuracy of the target signature in the diagnosis of prostate cancer was compared to the accuracy provided by other existing techniques, as shown in FIG. 5. The present invention proved to be the best method with an accuracy of 97.29% by using a target signature with only 2 biomarkers.

The details of the acronyms listed in the FIGS. 3 and 5 are: TSP (Top scoring pair), K-TSP (k-Top scoring pair), PAM (Prediction analysis of microarrays), DT (C4.5 decision trees).

The invention is also related to a computer program product comprising computer code arranged to be executed by processing means in order to carry out some or all of the above described methods when the processing means execute this computer code.

1. A method for discovering at least a biomarkers signature from biomarker data pools, the method comprising the steps of:
i) Generating a set of signatures fuzzy logic and evolutionary algorithms, each signature reciting a determined number of biomarkers;
ii) Selecting at least a target signature from said set of signatures by applying at least the following filters on said set of signatures:
a) a performance filter comprising:
setting a performance threshold for at least a performance criterion;
removing signatures having a value below said performance threshold for said performance criteria;
b) a frequency filter for sorting said set of signatures depending on the frequency of one biomarker within said set of signatures or the co-frequency of several biomarkers within the same signature among said set of signatures; and
c) a dominance filter comprising:
ranking the set of signatures depending on at least one performance criteria;
computing a dominance value for each signature, the dominance value being the number of signatures with a superior ranking for said performance criteria;
setting a dominance threshold and removing the signatures having a dominance value higher than said dominator threshold.

2. A method according to claim 1 wherein the target signature is selected by using successively at least one performance filter, at least one frequency filter and at least one dominance filter.

3. A method according to claim 1 wherein step ii) comprises:
applying several sequence of filters on the set of signatures, each filter sequence comprising at least one filter, each filter sequence being applied separately on the set of signatures so that each filter sequence provides at least one preselection of signature;
identifying at least one common signature, said common signature being at least one signature present in all the preselection;
combining the common signature(s);
applying at least one filter on the common signature(s).

4. A method according to claim 1 wherein step ii) comprises the successive steps of:
applying a first performance filter on the set of signatures;
applying at first frequency filter the signature isolated in the previous step to provide a first preselection of signature;
applying a first dominance filter independently on the signature isolated by the first performance filter to provide a second preselection of signature;
combining the signature listed in both the first preselection and the second preselection;
applying a second dominance filter on the signature isolated in the previous step;
applying a second performance filter on the signature isolated in the previous step;
applying a third performance filter on the signature isolated in the previous step;
applying an expert filter on the signature isolated in the previous step.

5. A method according to claim 1 wherein the target signature(s) comprises at least one rule.

6. A method according to claim 1 wherein the performance criteria is chosen among specificity, sensitivity, accuracy, positive and negative predictive values (PPV and NPV respectively), number of biomarker or rule per signature, area under the ROC curve (AUC), and average distance measurement (ADM).

7. A method according to claim 1 further comprising an expert filter applied by an expert in signature discovery to remove at least one biomarker from the target signature.

8. A method according to claim 1 wherein the application of one frequency filter comprises:
selecting at least one biomarker listed in the set of signatures;
removing the signature free from said selected biomarker.

9. A method according to claim 1 wherein the application of one frequency filter comprises:
computing a frequency of each biomarker in the set of signatures;
defining a frequency threshold for at least one biomarker;
removing the signature(s) comprising biomarker(s) with a frequency below said frequency threshold.

10. A method according to claim 1 wherein several iterations of the method are performed, each iteration providing a family of target signatures.

11. A method according to claim 1 wherein the data pools comprises biomedical data from sick and from healthy patients.

12. A method according to claim 1 wherein the data pools comprises data from sick and from healthy plants.

13. A method according to claim 1 wherein the data pools comprises data from sick and from healthy animals.

14. A method according to claim 1 wherein the data pools comprises data chosen amongst protein or nucleic acid measurements, physiological parameters such as age, weight, gender, or any clinical data.

15. A device for discovering at least a biomarkers signature from biomarker data pools, the device comprising:
A system combining fuzzy logic with evolutionary algorithms for generating a set of signatures each signature reciting a determined number of biomarker;
A filter system for selecting at least a target signature from said set of signatures, said filter system comprising at least the following filters:
a performance filter for sorting said set of signatures depending on at least one performance criteria;
a frequency filter for sorting said set of signatures depending on the frequency of one biomarker within said set of signatures or the co-frequency of several biomarkers within the same signature among said set of signatures;
a dominance filter, each signature has a dominance value so that the dominance filter is capable of sorting said set of signatures depending on the dominance value.

16. Use of a biomarker signature discovered by a method according to claim 1 for diagnosing, predicting or monitoring a disease.

17. Use according to claim 16, wherein the disease is chosen among plant disease, human disease, animal disease.

18. A computer program product comprising computer code executable by processing means in order to carry out the method of claim 1 when the computer code is executed.
